SEQUENCE LISTING (4 sequences)

SEQ ID NO: 1 - DNA, 26 bp, Legionella pneumophila
  misc_feature: Forward Primer
gtactaattggctgattgtcttgacc

SEQ ID NO: 2 - DNA, 21 bp, Legionella pneumophila
  misc_feature: Reverse Primer
cctggcgatgacctactttcg

SEQ ID NO: 3 - DNA, 35 bp, Legionella pneumophila
  misc_feature: Legionella pneomophilia specific probe
atcgtgtaaactctgactctttaccaaacctgtgg

SEQ ID NO: 4 - DNA, 22 bp, Legionella pneumophila
  misc_feature: Legionella spp. probe
atctcgaactcagaagtgaaac

(19) United States
(12) Patent Application Publication
(10) Pub. No.: US 2011/0318737 A1
(43) Pub. Date: Dec. 29, 2011

(54) REAL-TIME POLYMERASE CHAIN REACTION DETECTION OF LEGIONELLA PNEUMOPHILA AND DIFFERENTIATION FROM OTHER LEGIONELLA SPECIES

(75) Inventors: Robert F. Benson, Doraville, GA (US); Brian P. Holloway, Atlanta, GA (US); Karen A. McCaustland, Snellville, GA (US); Patrick Genyan Yang, Atlanta, GA (US)

(73) Assignee: The Government of the US as Represented by the Secretary of the Dept.of Health and Human Services, Atlanta, GA (US)

(21) Appl. No.: 13/140,922

(22) PCT Filed: Dec. 17, 2009
(86) PCT No.: PCT/US2009/068461

§ 371 Date: Sep. 12, 2011

Related U.S. Application Data
(60) Provisional application No. 61/138,727, filed on Dec. 18, 2008.

(51) Int. Cl. C12Q 1/68; C07H 21/04

(52) U.S. Cl. 435/6.11; 435/6.12; 536/24.32

(57) ABSTRACT

Materials and processes are provided for the detection of Legionella species in a sample. The inventive process includes exposing a sample to a forward primer and a reverse primer to yield an amplicon. The amplicon is detectable by at least one probe. The inventive process detects multiple species of Legionella bacteria or is specific for Legionella pneumophila. A kit is provided with inventive primers and probes for the detection of a Legionella species in a sample.

### RELATED APPLICATIONS

This application claims priority benefit of U.S. Provisional Application Ser. No. 61/138,727, filed 18 Dec. 2008; the contents of which are hereby incorporated by reference.

### GOVERNMENT INTEREST

The invention described herein may be manufactured, used, and licensed by or for the United States Government.

### FIELD OF THE INVENTION

The invention relates generally to a process of detecting Legionella species in real time in a fluid or solid sample; and in particular, to the detection of 23S-5S ribosomal intergenic spacer region with-real time polymerase chain reaction (PCR) that is capable of detecting all Legionella species and discriminating Legionella pneumophila from other Legionella species.

### BACKGROUND OF THE INVENTION

Legionella bacteria are ubiquitous in natural and man-made aqueous environments. To date, 52 Legionella spp. have been identified. Twenty-three of these species are associated with human diseases [1, 2]. Approximately 80-90% of the reported cases are attributed to L. pneumophila, however, all species may cause infection, especially in immunocompromised hosts [1, 3, 4].

Legionnaires Disease (LD) has no unique clinical or radiographical features [5, 6], which may lead to inappropriate therapy and a poor prognosis. Therefore, a validated and rapid diagnostic assay is of great importance. Current laboratory methods for confirming diagnosis of legionellosis consist of isolating Legionella bacteria by culture, detecting L. pneumophila serogroup 1 antigen in urine or seroconversion. Although these processologies offer good specificity, they primarily detect L. pneumophila. Non-pneumophila Legionella spp. may grow on buffered charcoal yeast extract (BCYE) media, but it usually takes about 1 to 2 weeks of incubation time for identification. Some strains such as Legionella-like amoebal pathogens are very fastidious and require amoebal co-culture [7], which is laborious and impractical for clinical diagnosis. Therefore, infections by non-pneumophila spp. are not often diagnosed.

To address these deficiencies, molecular assays that target the nucleic acid of Legionella have been developed, but their applications in clinical diagnosis are still limited. For example, the proportion of cases diagnosed by PCR and other genotypic processes in Europe from 1995-2004 accounts for less than 2% of LD (n=27,244) [8]. Reluctance in using PCR assays for diagnosis is partially due to: 1) post-amplification procedures that are laborious and time consuming and prone to carryover contamination and false positivity; and 2) limited assay optimization and validation.

Thus, there exists a need for a real-time PCR assay that is capable of rapidly detecting and differentiating L. pneumophila from 51 non-pneumophila species without the need for post-PCR manipulation. Such an assay preferably increases the detection rate of infection by non-pneumophila strains of Legionella and aid in early LD diagnosis.

### SUMMARY OF THE INVENTION

An inventive process includes detecting the presence of a Legionella species in a sample wherein a sample is exposed to a forward primer and a reverse primer under conditions suitable for polymerase chain reaction to yield an amplicon. The forward primer and reverse primer are preferably specific to a 23S-5S ribosomal intergenic spacer region of Legionella bacteria. The process includes detecting the amplicon indicative of the Legionella species.

Preferably, the inventive process uses a forward primer of SEQ ID NO: 1 and a reverse primer of SEQ ID NO: 2. Detection is preferably by real-time PCR, mass spectrometry, or HPLC. More preferably, detection is by real-time PCR. At least one detectable probe complimentary to the amplicon is provided that is either specific for Legionella pneumophila, or is generic to at least 52 known species of Legionella. Preferably, a probe has the sequence of either SEQ ID NO: 3 or SEQ ID NO: 4. The probe is interacted with the amplicon whereby detecting is by detecting the interaction between the probe and the amplicon.

An inventive process preferably detects multiple species of Legionella in a sample. Preferably, detecting is by detecting two or more differentiable probes. Detecting is preferably simultaneous detection of both probes.

A kit is provided including a forward primer with sequence SEQ ID NO: 1; a reverse primer with SEQ ID NO: 2; and a detectable probe specific for Legionella pneumophila or generic to the 52 known Legionella species. Preferably, the kit includes a probe that has the sequence of SEQ ID NO: 3, SEQ ID NO: 4, or a combination thereof.

### BRIEF DESCRIPTION OF THE DRAWINGS

FIG. 1A represents the inventive primers and probes complementary to the 23S-5S ribosomal intergenetic region of a Legionella species;

FIG. 1B represents the efficiency of amplification of the inventive assay;

FIG. 1C represents amplification of a specific site of Legionella in the presence of high levels of genomic DNA.

### DETAILED DESCRIPTION OF THE PREFERRED EMBODIMENTS

Materials and processes are provided for detecting the 23S-5S ribosomal intergenic spacer region conserved among all Legionella species. The current invention has utility as a composition and assay for the sensitive and rapid detection of Legionella pneumophila and all other Legionella spp. both in a laboratory and field setting as well as for diagnosis of LD in a subject. The detection of a Legionella spp. in real time accelerates clinical interventions in subjects and alleviation of conditions conductive to Legionella spp. growth.

The following definitional terms are used throughout the specification without regard to placement relative to these terms.

As used herein, the term “variant” defines either a naturally occurring genetic mutant of Legionella spp. or a recombinantly prepared variation of Legionella spp., each of which contain one or more mutations in its genome compared to the Legionella spp.

As used herein the term “species” is used synonymously with the abbreviation “spp.”

As used herein, the term “derivative” in the context of a non-proteinaceous derivative defines a second organic or inorganic molecule that is formed based upon the structure of a first organic or inorganic molecule. A derivative of an organic molecule includes, but is not limited to, a molecule modified, e.g., by the addition or deletion of a hydroxyl, methyl, ethyl, carboxyl or amine group. An organic molecule may also be esterified, alkylated and/or phosphorylated. A derivative also defined as a degenerate base mimicking a C/T mix such as that from Glen Research Corporation, Sterling, Va., illustratively LNA-dA or LNA-dT, or other nucleotide modification known in the art or otherwise. A nucleotide is optionally locked.

As used herein, the term “mutant” defines the presence of mutations in the nucleotide sequence of an organism as compared to a wild-type organism.

As used herein, the term “hybridizes under stringent conditions” describes conditions for hybridization and washing under which nucleotide sequences having at least 30%, 35%, 40%, 45%, 50%, 55%, 60%, 65%, 70%, 75%, 80%, 85%, 90%, or 95% identity to each other typically remain hybridized to each other. Such hybridization conditions are described in, for example but not limited to, Current Protocols in Molecular Biology, John Wiley & Sons, N.Y. (1989), 6.3.1 6.3.6.; Basic Methods in Molecular Biology, Elsevier Science Publishing Co., Inc., N.Y. (1986), pp. 75 78, and 84 87; and Molecular Cloning, Cold Spring Harbor Laboratory, N.Y. (1982), pp. 387 389, and are well known to those skilled in the art. A preferred, non-limiting example of stringent hybridization conditions is hybridization in 6× sodium chloride/sodium citrate (SSC), 0.5% SDS at about 68° C. followed by one or more washes in 2×SSC, 0.5% SDS at room temperature. Another preferred, non-limiting example of stringent hybridization conditions is hybridization in 6×SSC at about 45° C. followed by one or more washes in 0.2×SSC, 0.1% SDS at 50 to 65° C.

An “isolated” or “purified” nucleotide or oligonucleotide sequence is substantially free of cellular material or other contaminating proteins from the cell or tissue source from which the nucleotide is derived, or is substantially free of chemical precursors or other chemicals when chemically synthesized. The language “substantially free of cellular material” includes preparations of a nucleotide/oligonucleotide in which the nucleotide/oligonucleotide is separated from cellular components of the cells from which it is isolated or produced. Thus, a nucleotide/oligonucleotide that is substantially free of cellular material includes preparations of the nucleotide having less than about 30%, 20%, 10%, 5%, 2.5%, or 1%, (by dry weight) of contaminating material. When nucleotide/oligonucleotide is produced by chemical synthesis, it is preferably substantially free of chemical precursors or other chemicals, i.e., it is separated from chemical precursors or other chemicals which are involved in the synthesis of the protein. Accordingly, such preparations of the nucleotide/oligonucleotide have less than about 30%, 20%, 10%, or 5% (by dry weight) of chemical precursors or compounds other than the nucleotide/oligonucleotide of interest. In a preferred embodiment of the present invention, the nucleotide/oligonucleotide is isolated or purified.

As used herein, the term “isolated” as related to bacteria is a bacterial cell type which is separated from other organisms which are present in the natural source of the bacteria, e.g., biological material such as cells, blood, serum, plasma, saliva, urine, stool, sputum, nasopharyngeal aspirates, and so forth. The isolated bacteria are optionally used to infect a subject cell.

As used herein, the term “sample” is defined as material obtained from a biological organism, a tissue, cell, cell culture medium, or any medium suitable for mimicking biological conditions, or from the environment. Non-limiting examples include bronchoalveolar lavage fluid, bronchial aspirates, lung biopsies, post-mortem tissue specimens, sputum, saliva, gingival secretions, cerebrospinal fluid, gastrointestinal fluid, mucous, urogenital secretions, synovial fluid, blood, serum, plasma, urine, cystic fluid, lymph fluid, ascites, pleural effusion, interstitial fluid, intracellular fluid, ocular fluids, seminal fluid, mammary secretions, vitreal fluid, nasal secretions, throat or nasal materials, pleural effusion, water, soil, biological waste, cell culture media, or any other fluid or solid media. In a preferred embodiment, bacterial agents are contained in serum, whole blood, bronchoalveolar lavage fluid, bronchial aspirates, sputum, or nasal secretions.

As used herein, the term “medium” refers to any liquid or fluid biological sample in the presence or absence of bacteria. Non-limiting examples include buffered saline solution, cell culture medium, acetonitrile, trifluoroacetic acid, combinations thereof, or any other fluid recognized in the art as suitable for combination with bacteria or other cells, or for dilution of a biological sample or amplification product for analysis.

An inventive process illustratively includes exposing a sample to a forward primer and a reverse primer, wherein the forward primer and reverse primer are each specific to a 23S-5S ribosomal intergenic spacer region conserved among 52 known species of Legionella bacteria as listed in Table 1.

TABLE 1
Legionella spp.

Legionella adelaidensis
Legionella anisa
Legionella beliardensis
Legionella birminghamensis
Legionella bozemanae
Legionella brunensis
Legionella busanensis
Legionella cherrii
Legionella cincinnatiensis
Legionella drancourtii
Legionella drozanskii
Legionella dumoffii
Legionella erythra
Legionella fairfieldensis
Legionella fallonii
Legionella feeleii
Legionella geestiana
Legionella gormanii
Legionella gratiana
Legionella gresilensis
Legionella hackeliae
Legionella impletisoli
Legionella israelensis
Legionella jamestowniensis
Legionella jordanis
Legionella lansingensis
Legionella londiniensis
Legionella longbeachae
Legionella lytica
Legionella maceachernii
Legionella micdadei
Legionella moravica
Legionella nautarum
Legionella oakridgensis
Legionella parisiensis
Legionella pittsburghensis
Legionella pneumophila
Legionella pneumophila subsp. fraseri
Legionella pneumophila subsp. pascullei
Legionella pneumophila subsp. pneumophila
Legionella quateirensis
Legionella quinlivanii
Legionella rowbothamii
Legionella rubrilucens
Legionella sainthelensi
Legionella santicrucis
Legionella shakespearei
Legionella spiritensis
Legionella steigerwaltii
Legionella taurinensis
Legionella tucsonensis
Legionella wadsworthii
Legionella waitersii
Legionella worsleiensis
Legionella yabuuchiae

It is appreciated that other species, yet to be discovered, but with 23S-5S sequences recognized by the inventive primers are similarly detectable by the inventive process. The primers are exposed to sample under conditions conducive to a polymerase chain reaction so as to yield an amplicon. The inventive process also optionally includes providing a detectable probe complementary to the amplicon under conditions allowing the probe to interact with the amplicon to allow detection of the amplicon, and detecting the amplicon indicative of the Legionella species.

As used herein, the terms “subject” and “patient” are synonymous and refer to a single or multicellular organism illustratively including, but not limited to, a human or non-human animal, preferably a mammal including a human, monkey, ape, other upper and lower primates, horse, donkey, goat, rabbit, mouse, rat, guinea pig, hamster, or non-mammals illustratively including avian species and insects, and any inclusive or other organism capable of infection or transfection by or with Legionella spp. It is appreciated that a subject is illustratively a single cell.

The inventive process provides a rapid, specific, and sensitive assay for detection of Legionella spp. in samples by amplifying one or more nucleotide sequences that allow an investigator to identify and optionally to distinguish between species present in the sample by processes similar to the polymerase chain reaction (PCR). The invention is optionally used to distinguish L. pneumophila from other Legionella spp.

The present invention relates to the use of sequence information of Legionella for diagnostic processes. More particularly, the present invention provides a process for detecting the presence or absence of nucleic acid molecules of one or more Legionella species, natural or artificial variants, analogs, or derivatives thereof, in a biological sample. The process involves obtaining a biological sample from one or more various sources and contacting the sample with a compound or an agent capable of detecting a nucleic acid sequence of Legionella spp., natural or artificial variants, analogs, or derivatives thereof, such that the presence of Legionella, natural or artificial variants, analogs, or derivatives thereof, is detected in the sample. In a preferred embodiment, the presence of Legionella pneumophila, natural or artificial variants, analogs, or derivatives thereof, is detected in the sample by a real-time polymerase chain reaction (real-time PCR) using primers that are constructed based on a partial nucleotide sequence of the Legionella genome.

An inventive process illustratively uses forward and reverse primers operable to amplify at least a portion of the 23S-5S ribosomal intergenetic spacer region of Legionella spp. bacteria. In the inventive process an oligonucleotide forward primer with a nucleotide sequence complementary to a unique sequence in a Legionella nucleotide sequence such as the 23S-5S sequence (GenBank accession No: FJ444813.1), is hybridized to its complementary sequence and extended. Similarly, a reverse oligonucleotide primer complementary to a second strand of Legionella DNA in the same or an alternate region is hybridized and extended. This system provides amplification of specific nucleotide sequences and is operable for simultaneous or sequential detection systems.

In a preferred embodiment a forward primer is 5′-GTA CTA ATT GGC TGA TTG TCT TGA CC-3′ (SEQ ID NO: 1) and a reverse primer is 5′-CCT GGC GAT GAC CTA CTT TCG-3′ (SEQ ID NO: 2).

A preferred agent for detecting Legionella spp. nucleic acid sequences is a labeled nucleic acid probe capable of hybridizing thereto or to amplification products produced by PCR amplification of a region between a forward and reverse primer pair. Preferably, the nucleic acid probe is a nucleic acid molecule comprising nucleic acid sequence of 5′-ATCGTGTAAACTCTGACTCTTTACCAAACCTGTGG-3′ (SEQ ID NO: 3), or a derivative thereof, which sufficiently specifically hybridizes under stringent conditions to a Legionella nucleic acid sequence. The nucleic acid sequence SEQ ID NO: 3 is operable to distinguish Legionella pneumophila from other Legionella species.

Alternatively, a probe that recognizes more than one species of Legionella is used. Preferably, the probe is 5′ATCTCGAACTCAGAAGTGAAAC-3 (SEQ ID NO: 3), or a derivative thereof. More preferably the probe is of sequence 5′-ATCTC“G”AA“C”T“C”A“G”AA“G”T“G”AAAC-3′ (SEQ ID NO: 4) where (“ ”) denotes a locked nucleic acid, which sufficiently specifically hybridizes under stringent conditions to a Legionella nucleic acid sequence.

It is appreciated that a probe is preferably labeled to aid in detection. In a preferred embodiment, SEQ ID NO: 3 is modified as 5′-CalOrg-ATCGTGTAAACTCTGACTCTTTACCAAACCTGTGG-3′BHQ (SEQ ID NO: 3). Preferably, SEQ ID NO: 4 is modified to include 5′-FAM ATCTC“G”AA“C”T“C”A“G”AA“G”T“G”AAAC-3′BHQ. (SEQ ID NO: 4).

BHQ in the above sequences denotes a quencher such as a black hole quencher 1, 2, 3, 4, or the like (Eurogentec). It is appreciated the BHQ is readily replaced by another quencher such as QSY-7 or others conventional to the art. FAM denotes 6 FAM (Glen Research) with a formal name of [(3′,6′-dipivaloylfluoresceinyl)-6-carboxamidohexyl]-1-O-(2-cyanoethyl)-(N,N-diisopropyl)-phosphoramidite. CalOrg is Cal Fluor Orange 560 and is readily substituted with other fluorophores such as HEX, VIC, AlexaFluor 495 or 590, Cascade Blue, Marina Blue, Pacific Blue, Oregon Green, Rhodamine, Fluoroscein, TET, VIC, HEX, Cy5, Cy3, Tetramethylrhodamine, or the like.

Legionella spp. is detected by culture, urine antigen test, seroconversion, real-time polymerase chain reaction (PCR), other nucleic acid based assays, mass spectrometry, high pressure liquid chromatography (HPLC), or PCR detection such as by amplification of genetic sequences by primers of SEQ ID NOs: 1-2.

Also, provided is a diagnostic assay process for detection of Legionella spp. infection in a patient wherein a sample from a patient suspected of being infected with Legionella spp. is exposed to a forward primer and a reverse primer and the 23S-5S ribosomal intergenetic spacer region is detected.

Real-time PCR is preferably used to detect Legionella spp. using a probe of SEQ ID NO: 3 or 4 wherein the probe is hybridized preferably under conditions suitable for a polymerase chain reaction; producing a first detection signal from the probe hybridized to the amplicon or the base genetic sequence. As such, the process diagnoses Legionella spp. infection in a human. Optionally, the first detection signal is compared to a control detection signal from a Legionella spp. calibrator extracted in parallel to the sample. The calibrator is preferably a known amount of Legionella spp. and a known amount of a medium similar to the sample.

The detection signal indicating the presence of at least one species of Legionella in the sample is optionally confirmed by sequencing of macrophage infectivity potentiator (mip) gene.

A process for detecting Legionella spp. is provided that uses a forward primer with sequence SEQ ID NO: 1; a reverse primer with SEQ ID NO: 2; and two probes. The probe that has the sequence SEQ ID NO: 3 specific for Legionella pneumophila and a probe that has the sequence SEQ ID NO: 4 recognizing the 52 known species of Legionella spp.

The instant inventive processes are amenable to use for diagnosis of Legionella infection in a subject.

The term “nucleotide” is intended to mean a base-sugar-phosphate combination either natural or synthetic, linear, circular and sequential arrays of nucleotides and nucleosides, e.g. cDNA, genomic DNA, RNA, oligonucleotides, oligonucleosides, and derivatives thereof. Included in this definition are modified nucleotides which include additions to the sugar-phosphate groups or to the bases.

A sample is preferably a fluidic sample. Illustratively, a fluidic sample such as serum or cell lysate is diluted in a buffered saline solution suitable for assay of a Legionella species. Alternatively, a sample is solid wherein a suspension is created in a buffered saline solution or the solid is dissolved in a solvent such as lysis buffer. An illustrative example of operative buffered solutions are 50 mM Tris-HCl,10 mM MgCl₂, 100 mM NaCl, pH 8.0 or 25 mM Tris/HCl, pH 7.6, 25 mM KCl, 5 mM MgCl₂. It is appreciated that other buffered or non-buffered solutions are similarly operable. Other buffers operable are illustratively, HEPES, Tris, phosphate, carbonate, imidizole, acetate, or any other buffer known in the art. Salts and other cations are further operable in the invention. (see e.g. Endo, Y, et al, J Biol Chem, 1987; 262:8128-30.) Preferably, magnesium ions are included in a buffer or solution. Endo, Y, J Biol Chem, 1988; 263:8735-8739. More preferably, magnesium is between 5 and 15 mM.

The inventive process includes a polymerization reaction. The polymerization reaction is performed by a nucleic acid polymerizing enzyme that is illustratively a DNA polymerase, RNA polymerase, reverse transcriptase, mixtures thereof, or other polymerases known in the art. It is further appreciated that accessory proteins or molecules are present to form the replication machinery. In a preferred embodiment the polymerizing enzyme is a thermostable polymerase or thermodegradable polymerase. Use of thermostable polymerases is well known in the art such as Taq polymerase available from Invitrogen Corporation, Carlsbad, Calif. Thermostable polymerases allow a polymerization reaction to be initiated or shut down by a change in temperature or other condition in the sample without destroying activity of the polymerase.

The process of the present invention optionally involves a real-time PCR assay that is preferably quantitative. In a preferred embodiment, the quantitative PCR used in the present invention is TaqMan assay (Holland et al., PNAS 88(16):7276 (1991)). It is appreciated that the current invention is amenable to performance on other real-time PCR systems and protocols that use alternative reagents illustratively including, but not limited to Molecular Beacons probes, Scorpion probes, multiple reporters for multiplex PCR, combinations thereof, or other DNA detection systems.

The assays are performed on an instrument designed to perform such assays, for example those available from Applied Biosystems (Foster City, Calif.). In more preferred specific embodiments, the present invention provides a real-time quantitative PCR assay to detect the presence of one or more Legionella species, natural or artificial variants, analogs, or derivatives thereof, in a biological sample by subjecting the Legionella nucleic acid from the sample to PCR reactions using specific primers, and detecting the amplified product using a probe. In preferred embodiments, the probe is a TaqMan probe which consists of an oligonucleotide with a 5′-reporter dye and a 3′-quencher dye.

Optionally, a fluorescent reporter dye, such as FAM dye (illustratively 6-carboxyfluorescein), is covalently linked to the 5′ end of the oligonucleotide probe. Other dyes illustratively include TAMRA, AlexaFluor dyes such as AlexaFluor 495 or 590, Cascade Blue, Marina Blue, Pacific Blue, Oregon Green, Rhodamine, Fluoroscein, TET, HEX, Cy5, Cy3, Quasar670, and Tetramethylrhodamine. Each of the reporters is optionally quenched by a dye at the 3′ end or other non-fluorescent quencher. Quenching molecules are suitably matched to the fluorescence maximum of the dye. Any suitable fluorescent probe for use in real-time PCR detection systems is illustratively operable in the invention. Similarly, any quenching molecule for use in real-time PCR systems is illustratively operable. In a preferred embodiment a 6-carboxyfluorescein reporter dye is present at the 5′-end and matched to Black Hole Quencher (BHQ1, Biosearch Technologies, Inc., Novato, Calif.). The fluorescence signals from these reactions are captured at the end of extension steps as PCR product is generated over a range of the thermal cycles, thereby allowing the quantitative determination of the bacterial load in the sample based on an amplification plot.

It is appreciated that other detection systems, techniques, and labels are operative herein. Illustratively, a probe is labeled with a radioactive marker. Illustrative radioactive labels include ³H, ¹³C, ³²P, ¹²⁵I, ¹³¹I, ²²Na, ⁵¹Cr, and other radioactive labels known in the art.

The Legionella nucleic acid sequences are optionally amplified before being detected. The term “amplified” defines the process of making multiple copies of the nucleic acid from a single or lower copy number of nucleic acid sequence molecule. The amplification of nucleic acid sequences is preferably carried out in vitro by biochemical processes known to those of skill in the art. The amplification agent is optionally any compound or system that will function to accomplish the synthesis of primer extension products, including enzymes. Suitable enzymes for this purpose include, for example, E. coli DNA polymerase I, Taq polymerase, Klenow fragment of E. coli DNA polymerase I, T4 DNA polymerase, AmpliTaq Gold DNA Polymerase from Applied Biosystems, other available DNA polymerases, reverse transcriptase (preferably iScript RNase H+ reverse transcriptase), ligase, and other enzymes, including heat-stable enzymes (i.e., those enzymes that perform primer extension after being subjected to temperatures sufficiently elevated to cause denaturation). In a preferred embodiment, the enzyme is hot-start iTaq DNA polymerase from Bio-rad (Hercules, Calif.). Suitable enzymes will facilitate combination of the nucleotides in the proper manner to form the primer extension products that are complementary to each mutant nucleotide strand. Generally, the synthesis is initiated at the 3′-end of each primer and proceed in the 5′-direction along the template strand, until synthesis terminates, producing molecules of different lengths. It is appreciated that amplification agents that initiate synthesis at the 5′-end and proceed in the other direction, using the same process as described above are similarly operable. In any event, the process of the invention is not to be limited to the embodiments of amplification described herein.

One process of in vitro amplification optionally used according to this invention, is the polymerase chain reaction (PCR) described in U.S. Pat. Nos. 4,683,202 and 4,683,195. The term “polymerase chain reaction” refers to a process for amplifying a DNA base sequence using a heat-stable DNA polymerase and two oligonucleotide primers, one complementary to the (+)-strand at one end of the sequence to be amplified and the other complementary to the (−)-strand at the other end. Because the newly synthesized DNA strands can subsequently serve as additional templates for the same primer sequences, successive rounds of primer annealing, strand elongation, and dissociation produce rapid and highly specific amplification of the desired sequence. Many polymerase chain processes are known to those of skill in the art and are optionally used in the process of the invention. For example, DNA is optionally subjected to 30 to 35 cycles of amplification in a thermocycler as follows: 95° C. for 30 sec, 52 to 60° C. for 1 min, and 72° C. for 1 min, with a final extension step of 72° C. for 5 min. For another example, DNA is subjected to 35 polymerase chain reaction cycles in a thermocycler at a denaturing temperature of 95° C. for 30 sec, followed by varying annealing temperatures ranging from 54 to 58° C. for 1 min, an extension step at 70° C. for 1 min, with a final extension step at 70° C. for 5 min. The parameters of PCR cycling times and number of steps is dependent on the primer pair, their melting temperature, and other considerations obvious to those known in the art. It is appreciated that optimizing PCR parameters for various probe sets is well within the skill of the art and is performed as mere routine optimization.

The primers or probes for use in amplifying the nucleic acid sequences of Legionella are illustratively prepared using any suitable process, such as conventional phosphotriester and phosphodiester processes or automated embodiments thereof so long as the primers or probes are capable of hybridizing to the nucleic acid sequences of interest. One process for synthesizing oligonucleotides on a modified solid support is described in U.S. Pat. No. 4,458,066. The exact length of primer or probe will depend on many factors, including temperature, buffer, and nucleotide composition. The primer generally primes the synthesis of extension products in the presence of the inducing agent for amplification.

Primers used according to the process of the invention are complementary to each strand of nucleotide sequence to be amplified. The term “complementary” means that the primers hybridize with their respective strands under conditions that allow the agent for polymerization to function. In other words, the primers hybridize with Legionella sequences(s) and permit amplification of the nucleotide sequence. Preferably, the 3′ terminus of the primer that is extended is perfectly base paired with the complementary flanking strand. Preferably, probes possess nucleotide sequences complementary to one or more strands of the amplification product such as from 23S-5S. More preferably, the primers and probes are complementary to genetic sequences specific to Legionella pneumophila. Most preferably, primers contain the nucleotide sequences of SEQ ID Nos: 1 and 2. It is appreciated that the complement of the aforementioned primer and probe sequences are similarly suitable for use in the invention. It is further appreciated that oligonucleotide sequences that hybridize with the inventive primer and probes are also similarly suitable. Finally, multiple positions are available for hybridization on the Legionella genome and will be also suitable for hybridization with a probe when used with the proper forward and reverse primers.

Those of ordinary skill in the art will know of various amplification processes that are optionally utilized to increase the copy number of target nucleic acid sequence. The nucleic acid sequences detected in the process of the invention are optionally further evaluated, detected, cloned, sequenced, and the like, either in solution or after binding to a solid support, by any process usually applied to the detection of a specific nucleic acid sequence such as another polymerase chain reaction, oligomer restriction (Saiki et al., BioTechnology 3:1008 1012 (1985)), allele-specific oligonucleotide (ASO) probe analysis (Conner et al., PNAS 80: 278 (1983)), oligonucleotide ligation assays (OLAs) (Landegren et al., Science 241:1077 (1988)), RNase Protection Assay and the like. Molecular techniques for DNA analysis have been reviewed (Landegren et al, Science 242:229 237 (1988)). Following DNA amplification, the reaction product is optionally detected by Southern blot analysis, with or without using radioactive probes. In such a process, for example, a small sample of DNA containing the nucleic acid sequence obtained from the tissue or subject is amplified, and analyzed via a Southern blotting technique. The use of non-radioactive probes or labels is facilitated by the high level of the amplified signal. In one embodiment of the invention, one nucleoside triphosphate is radioactively labeled, thereby allowing direct visualization of the amplification product by autoradiography. In another embodiment, amplification primers are fluorescently labeled and run through an electrophoresis system. Visualization of amplified products is by laser detection followed by computer assisted graphic display, without a radioactive signal.

Other methods of detecting amplified oligonucleotide illustratively include gel electrophoresis, mass spectrometry, liquid chromatography, fluorescence, luminescence, gel mobility shift assay, fluorescence resonance energy transfer, nucleotide sequencing, enzyme-linked immunoadsorbent assay, high performance liquid chromatography, ultra-high performance liquid chromatography, enzyme-linked immunoadsorbent assay, real-time PCR, affinity chromatography, immunoenzymatic methods (Ortiz, A and Ritter, E, Nucleic Acids Res., 1996; 24:3280-3281), streptavidin-conjugated enzymes, DNA branch migration (Lishanski, A, et al., Nucleic Acids Res., 2000; 28(9):e42), enzyme digestion (U.S. Pat. No. 5,580,730), colorimetric methods (Lee, K., Biotechnology Letters, 2003; 25:1739-1742), or combinations thereof.

The term “labeled” with regard to the probe is intended to encompass direct labeling of the probe by coupling (i.e., physically linking) a detectable substance to the probe, as well as indirect labeling of the probe by reactivity with another reagent that is directly labeled. Examples of indirect labeling include detection of a probe using a fluorescently labeled antibody and end-labeling or centrally labeling of a DNA probe with biotin such that it can be detected with fluorescently labeled streptavidin. The detection method of the invention is optionally used to detect RNA (particularly mRNA) or genomic nucleic acid in a sample in vitro as well as in vivo. For example, in vitro techniques for detection of nucleic acid include northern hybridizations, in situ hybridizations, RT-PCR, real-time PCR, and DNase protection.

The size of the primers used to amplify a portion of the nucleic acid sequence of Legionella is optionally at least 5, and often 10, 15, 20, 25, or 30 nucleotides in length. Preferably, the GC ratio is above 30%, 35%, 40%, 45%, 50%, 55%, or 60% so as to prevent hair-pin structure on the primer. Furthermore, the amplicon is preferably sufficiently long enough to be detected by standard molecular biology methodologies. The forward primer is preferably longer than the reverse primer. Techniques for modifying the Tₘ of either primer are operable herein. An illustrative forward or reverse primer contains LNA-dA, LNA-dC, LNA-dG, and LNA-dT (Glen Research Corporation) optionally to match Tₘ with a corresponding alternate primer.

Accuracy of the base pairing in the preferred embodiment of DNA sequencing is provided by the specificity of the enzyme. Error rates for Taq polymerase tend to be false base incorporation of 10⁻⁵ or less. (Johnson, Annual Reviews of Biochemistry, 1993: 62:685-713; Kunkel, Journal of Biological Chemistry, 1992; 267:18251-18254). Specific examples of thermostable polymerases illustratively include those isolated from Thermus aquaticus, Thermus thermophilus, Pyrococcus woesei, Pyrococcus furiosus, Thermococcus litoralis and Thermotoga maritima. Thermodegradable polymerases illustratively include E. coli DNA polymerase, the Klenow fragment of E. coli DNA polymerase, T4 DNA polymerase, T7 DNA polymerase and other examples known in the art. It is recognized in the art that other polymerizing enzymes are similarly suitable illustratively including E. coli, T7, T3, SP6 RNA polymerases and AMV, M-MLV, and HIV reverse transcriptases.

The polymerases are optionally bound to the primer. When the genetic material of Legionella is a single-stranded DNA molecule due to heat denaturing the polymerase is bound at the primed end of the single-stranded nucleic acid at an origin of replication. A binding site for a suitable polymerase is optionally created by an accessory protein or by any primed single-stranded nucleic acid.

It is further appreciated that the proteinaceous material of the polymerization enzyme in the case of a DNA polymerase is optionally immobilized on a solid support surface either reversibly or irreversibly. For example, RNA polymerase was successfully immobilized on an activated surface without loss of catalytic activity. Yin et al., Science, 1995; 270: 1653-57. Alternatively, an antibody antigen pair is utilized to bind a polymerase enzyme to a support surface whereby the support surface is coated with an antibody that recognizes an epitope on the protein antigen. When the antigen is introduced into the reaction chamber it is reversibly bound to the antibody and immobilized on the support surface. A lack of interference with catalytic activity in such a process has been reported for HIV reverse transcriptase. Lennerstrand, Analytical Biochemistry, 1996; 235:141-152. Additionally, DNA polymerase immobilization has been reported as a functional immobilization process in Korlach et al., U.S. Pat. No. 7,033,764 B2. Finally, any protein component is optionally biotinylated such that, illustratively, a biotin streptavidin interaction is created between the support surface and the target immobilized antigen.

A real-time PCR assay system is employed such as the TAQMAN system available from Applied Biosystems (Foster City, Calif.) or the iCycler iQ real-time detection system (Bio-Rad, Hercules, Calif.). It is appreciated that a probe based process, intercalator-based process, or other process known in the art are operable herein. Suitable probes target the amplicon region of Legionella and are optionally between 15 and 60 nucleotides long, are unique to the target sequence, are not prone to dimerization, and do not possess repeat regions. Processes of probe design and considerations for use are recognized in the art.

In a further embodiment detection of PCR products is achieved by mass spectrometry. Mass spectrometers are prevalent in the clinical laboratory. Similar to fluorescence based detection systems mass spectrometry is capable of simultaneously detecting multiple amplification products for a multiplexed and controlled approach to accurately quantifying components of biological or environmental samples.

Multiple mass spectrometry platforms are suitable for use in the invention illustratively including matrix assisted laser desorption ionization time of flight mass spectrometry (MALDI), electrospray mass spectrometry, electrospray ionization-Fourier transform ion cyclotron resonance mass spectrometry (ESI-FTICR), multi-stage mass spectrometry fragmentation analysis (MS/MS), mass spectrometry coupled with liquid chromatography such as high performance liquid chromatography mass spectrometry (HPLC) and ultra performance liquid chromatography isotope dilution tandem mass spectrometry (UPLC-ID/MS/MS), and variations thereof.

Optionally, multiple amplification products are simultaneously produced in a PCR reaction that are then available for simultaneous detection and quantification. Thus, multiple detection signals are inherently produced or emitted that are separately and uniquely detected in one or more detection systems. It is appreciated that multiple detection signals are optionally produced in parallel. Preferably, a single biological sample is subjected to analysis for the simultaneous or sequential detection of Legionella genetic sequences. It is appreciated that two or more independent or overlapping sequences are simultaneously or sequentially measured in the instant inventive process. Oligonucleotide matched primers (illustratively SEQ ID NOs: 1 and 2) are simultaneously or sequentially added and the biological sample is subjected to proper thermocycling reaction parameters. For detection by mass spectrometry a single sample of the amplification products from each gene are simultaneously analyzed allowing for rapid and accurate determination of the presence of Legionella. Optionally, analysis by real-time PCR is employed capitalizing on multiple probes with unique fluorescent signatures. Thus, each gene or other genetic sequence is detected without interference by other amplification products. This multi-target approach increases confidence in quantification and provides for additional internal control.

To increase confidence and to serve as an internal or external control, a purified or otherwise characterized Legionella spp. solution is used as a sample. By amplification of a single sample with known quantities of Legionella spp. or of a set of samples representing a titration of Legionella spp., the quantity of Legionella spp. in the unknown biological sample is determined. Preferably, the purified and characterized Legionella spp. solution is analyzed in parallel with the unknown sample to reduce inter assay error or to serve as a standard curve for quantification of unknown Legionella spp. in the sample.

The invention also encompasses a kit for detecting the presence of Legionella spp. in a sample. The kit, for example, includes oligonucleotides capable of detecting Legionella pneumophila or other Legionella spp. in a test sample and, in certain embodiments, for quantifying Legionella pneumophila and other Legionella spp. in the sample.

For oligonucleotide-based kits, the kit includes, for example: (1) a pair of primers (one forward and one reverse) useful for amplifying a nucleic acid molecule synthesized in the presence of Legionella spp./pneumophila; and/or (2) a probe operable for detecting amplified oligonucleotide in response to Legionella spp./pneumophila. The kit also illustratively includes a buffering agent, a preservative, or a protein stabilizing agent. It is appreciated that a kit is optionally as simple as a substrate for addition to a sample or as complex as all reagents, enzymes, oligonucleotides, and detection apparatus' necessary for full detection and quantification of Legionella spp./pneumophila in a sample. The kit optionally includes components necessary for detecting the detectable agent (e.g. synthesized complementary strand). The kit also optionally contains a control sample or a series of control samples that is assayed and compared to the test sample contained. Each component of the kit is optionally enclosed within an individual container(s) and all of the various containers are optionally enclosed within a single package along with instructions for use.

Ancillary reagents are any signal producing system materials for detection of Legionella spp. in any suitable detection process such as real time PCR, ELISA, mass spectrometry, Southern blot, immunoprecipitation, HPLC, UHPLC, or other process known in the art. In a preferred embodiment, a kit illustratively includes a microtiter plate or other support or chamber such as an collection tube sealable or not sealable, control sample containing Legionella spp., buffer, swab or other sample collection devices, control reagents such as competing or unlabelled reagents, control substrate and relevant primers and probes, and other materials and reagents for detection. The kit optionally includes instructions printed or in electronic form and customer support contact information. Probes in a signal producing system or otherwise are optionally labeled with a fluorophore, biotin, peroxidase, or other enzymatic or non-enzymatic detection label such as a radioactive label or otherwise.

The components of the kit are any of the reagents described above or other necessary and non-necessary reagents known in the art for solubilization, detection, washing, storage, or other need for in an assay kit.

Methods involving conventional biological techniques are described herein. Such techniques are generally known in the art and are described in detail in methodology treatises such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992.

Various aspects of the present invention are illustrated by the following non-limiting examples. The examples are for illustrative purposes and are not a limitation on any practice of the present invention. It is understood that variations and modifications can be made without departing from the spirit and scope of the invention. While the examples are generally directed to mammalian cells, tissue, fluids, or subjects, a person having ordinary skill in the art recognizes that similar techniques and other techniques know in the art readily translate the examples to other subjects. Reagents illustrated herein are commonly cross reactive between mammalian species or alternative reagents with similar properties are commercially available, and a person of ordinary skill in the art readily understands where such reagents may be obtained.

### Example 1

### Bacterial Strains

Non-Legionella bacteria and viruses are used to test specificity of real-time PCR to detect nucleic acid sequences including Bordetella pertussis, Bordetella parapertussis, Bordetella holmesii, Bordetella bronchiseptica, Corynebacterium diphtheriae, Candida albicans, Chlamydophila pneumoniae, Chlamydia psittaci, Chlamydophila trachomatis, Escherichia coli, Haemophilus influenzae, Mycoplasma pneumoniae, Mycoplasma hominis, Mycoplasma genitalium, Ureaplasma urealyticum, Mycoplasma arginini, Mycoplasma buccale, Mycoplasma faucium, Mycoplasma fermentans, Mycoplasma hyorhinis, Mycoplasma lipophilum, Mycoplasma orale, Mycoplasma penetrans, Mycoplasma pirum, Mycoplasma salivarium, Moraxella catarrhalis, Mycobacterium tuberculosis, Neisseria meningitidis, Neisseria elongata, Pseudomonas aeruginosa, Streptococcus pneumoniae, Streptococcus pyogenes, Streptococcus salivarius, Streptococcus oligofermentans, Streptococcus sustralis, Streptococcus vestibularis, Streptococcus sinesis, Streptococcus gordonii, Streptococcus agalactiae, Streptococcus peroris, Streptococcus sanguinis, Streptococcus parasanguinis, Streptococcus infantis, Streptococcus mitis, Streptococcus cristatus, Streptococcus oxalis, Klebsiella pneumoniae, Staphylococcus aureus, Staphylococcus epidermidis, Influenza A (H1, H3, H5), Influenza B, SARS, Respiratory syncytial virus (A, B), Human parainfluenza viruses (1, 2, 3), Human metapneumovirus, and Adenovirus. Each is cultured with appropriate agar, cells and medium.

The inventive real-time PCR assay is also analyzed using Legionella strains that are grown on BCYE agar at 35□C with 2.5% CO₂ for 48-72 hours. Legionella spp. listed in Table 1 are analyzed for the ability of the assay to amplify the target sequence. The incubation period for the culture of human specimens is 7 days. The bacteria derived from a single colony are harvested in water and subjected to genomic DNA purification.

### Example 2

### Primer and Probes

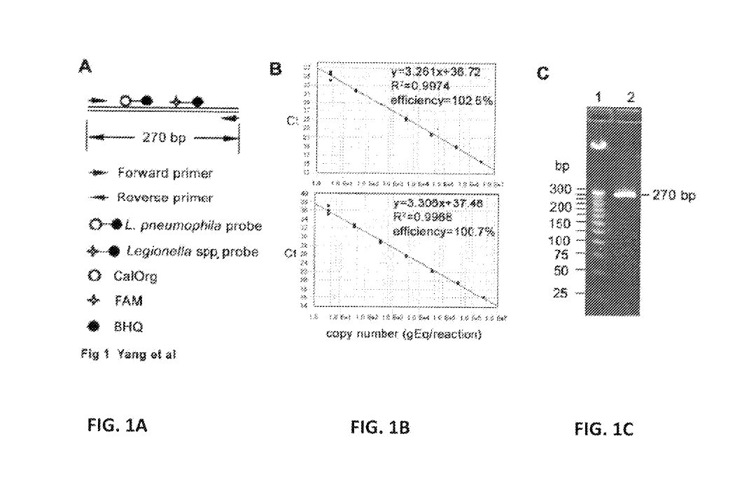

Primers are designed to the 23S-5S ribosomal intergenic spacer region conserved for all of Legionella spp. One set of primers and two probes are designed within the same region providing for singleplex dual color real-time PCR analyses. (FIG. 1A) The sequences of forward and reverse primers are 5′-GTACTAATTGGCTGATTGTCTTGACC-3′ (SEQ ID NO: 1), and 5′-CCTGGCGATGACCTACTTTCG-3′(SEQ ID NO: 2), respectively. A first probe specific for L. pneumophila (5′-CalOrg-ATC GTG TAA ACT CTG ACT CTT TAC CAA ACC TGT GG-3′BHQ), is synthesized as well as a second probe that recognizes all known Legionella spp. (5′-FAM ATC TC“G” AA“C”T“C”A “G”AA “G”T“G”AAA C-3′BHQ (SEQ ID NO: 4) (“ ” denotes lock nucleic acid). All PCR reactions are performed in triplicate using the AgPath-ID One-Step RT-PCR kit (Cat# AM1005, AppliedBiosystems Inc.) on 7900HT real-time PCR system (AppliedBiosystems Inc.).

### Example 3

### Real-time PCR

PCR reactions are performed using the primers of Example 2 at working concentrations from 25 nM to 200 nM at 2-fold intervals. The lowest Ct is observed with forward/reverse primers at 100 nM and 200 nM, respectively. The probes are titrated similarly from 50 nM to 600 nM. Use of Legionella spp. probe at concentration of 200 nM produces the best performance. Each Legionella species of Table 1 tested is readily detectable by the assay. The L. pneumophila specific probe shows superior performance when used at 400 nM.

Linear regression analysis of the standard curves reveal linear correlation present over 7 orders of magnitude between the Ct value and the copy number of the Legionella genome used in the inventive assay (R²=0.997). The efficiency of amplification is 100.7% and 102.6% for L. pneumophila and the genus wide assays, respectively (FIG. 1B). The primers target to the specific sites even in the presence of high concentration of Legionella genomic DNA (FIG. 1C).

The analytical sensitivity is assessed by amplifying L. pneumophila serogroup 1 DNA at a concentration of 3 gEq per PCR reaction. All samples using the Legionella spp. probe (n=20, Ct=34.59±0.76, CV=2.22%) are positive and 95% of the samples are positive with the L. pneumophila probe (n=20, Ct=35.51±0.96, CV=2.69%). In contrast, no signals are detected from no-template controls (NTCs, n>100) after 40 cycles of amplification. The lower limit of detection (LLOD) of the assay with these specific primers is ˜3 gEq per PCR reaction. For live bacteria, the assay detects L. pneumophila serogroup 1 at 7.5 CFU/mL (n=30, for the Legionella spp. probe: Ct=33.83±0.52, coefficient of variation (CV)=1.53%; for the L. pneumophila probe: Ct=37.05±0.92, CV=2.48%). No amplification is detected in the no-template controls (n=10).

### Example 4

### Examination of Infection in Clinical Samples

149 clinical specimens from 67 patients with respiratory disease due to possible infection by Legionella are collected and subjected to analyses by the assay of Example 3. All samples are cultured on BCYE or selective media. The inventive assay detects Legionella spp. in all of the 39 culture-positive specimens. The inventive assay also detects Legionella in 27 out of 110 culture-negative samples demonstrating the improved detection ability of the inventive assay over prior art methods of clinical diagnosis such as culture. The presence of Legionella spp. in these RT-PCR positive specimens is confirmed by sequencing the amplicon or by amplifying the mip gene target as described by Ratcliff, R. M., et al., J. Clin. Microbiol., 1998; 36(6): p. 1560-1567, the contents of which are incorporated herein by reference. 15 of the culture negative samples are positive for L. pneumophila, 2 were positive for L. longbeachae, one is positive for L. cincinnatiensis and one is positive for L. micdadei.

Throughout this application, various publications are referenced. The disclosures of these publications in their entireties are hereby incorporated by reference into this application for the entire teaching of each publication as related to this application and to more fully describe the state of the art to which this invention pertains.

The foregoing description is illustrative of particular embodiments of the invention, but is not meant to be a limitation upon the practice thereof. The following claims, including all equivalents thereof, are intended to define the scope of the invention.

We claim:

### PRIOR ART REFERENCES RELEVANT TO THE INVENTION

1. Muder Robert, R. and L. Yu Victor, Infection due to Legionella species other than L. PNEUMOPHILA. Clinical infectious diseases: an official publication of the Infectious Diseases Society of America, 2002. 35(8)): p. 990-8.
2. Berger, P., et al., Ameba-associated microorganisms and diagnosis of nosocomial pneumonia. Emerging infectious diseases, 2006. 12(2): p. 248-55.
3. McNally, C., et al., Potential importance of Legionella species as etiologies in community acquired pneumonia (CAP). Diagn Microbiol Infect Dis, 2000. 38(2): p. 79-82.
4. Roig, J., M. Sabria, and L. Pedro-Botet Maria, Legionella spp.: community acquired and nosocomial infections. Current opinion in infectious diseases, 2003. 16(2): p. 145-51.
5. Gupta, S. K., T. F. Imperiale, and G. A. Sarosi, Evaluation of the Winthrop-University Hospital criteria to identify Legionella pneumonia. Chest, 2001. 120(4): p. 1064-71.
6. Fields, B S., Benson R. B., and Richard E B., Legionella and Legionnaires' disease: 25 years of investigation. Clinical microbiology reviews, 2002. 15(3): p. 506-26.
7. Adeleke, A. A., et al., Legionella drozanskii sp. nov., Legionella rowbothamii sp. nov. and Legionella fallonii sp. nov.: three unusual new Legionella species. International journal of systematic and evolutionary microbiology, 2001. 51(Pt 3): p. 1151-60.
8. Diederen, B. M., Legionella spp. and Legionnaires' disease. J Infect, 2008. 56(1): p. 1-12.
9. Ratcliff, R. M., et al., Sequence-based classification scheme for the genus Legionella targeting the mip gene. Journal of Clinical Microbiology, 1998. 36(6): p. 1560-1567.
10. Domínguez, J. A., et al., Comparison of the BinaxLegionella urinary antigen enzyme immunoassay (EIA) with the Biotest □Legionella □ urine antigen EIA for detection of Legionella antigen in both concentrated and nonconcentrated urine samples. 1998. 36(9): p. 2718-2722.
11. Benin, A. L., R. F. Benson, and R. E. Besser, Trends in legionnaires disease, 1980-1998: declining mortality and new patterns of diagnosis. Clin Infect Dis, 2002. 35(9): p. 1039-46.
12. Phares, C. R., et al., Epidemiology of severe pneumonia caused by Legionella longbeachae, Mycoplasma pneumoniae, and Chlamydia pneumoniae: 1-year, population-based surveillance for severe pneumonia in Thailand. Clin Infect Dis, 2007. 45(12): p. e147-55.
13. van Belkum, A., M. Struelens, and W. Quint, Typing of Legionella PNEUMOPHILA Strains by Polymerase Chain Reaction-Mediated DNA Fingerprinting. Journal of Clinical Microbiology, 1993. 31(8): p. 2198-2200.
14. Salloum, G., et al., Identification of Legionella species by ribotyping and other molecular methods. Research in microbiology, 2002. 153(10): p. 679-86.
15. Grattard, F., et al., Analysis of the genetic diversity of Legionella by sequencing the 23S-5S ribosomal intergenic spacer region: from phylogeny to direct identification of isolates at the species level from clinical specimens. Microbes and infection/Institut Pasteur, 2006. 8(1): p. 73-83.
16. Fry, N. K., et al., The use of 16S ribosomal RNA analyses to investigate the phylogeny of the family Legionella ceae. 1991. 137: p. 1215-1222.
17. Ezaki, T., et al., Evaluation of the microplate hybridization method for rapid identification of Legionella species. European Journal Of Clinical Microbiology & Infectious Diseases, 1990. 9: p. 213-217.
18. Fain, J. S., et al., Rapid diagnosis of Legionella infection by a nonisotopic in situ hybridization method. 1991. 95: p. 719-724.
19. Buchbinder, S., et al., Mixed lung infection by Legionella PNEUMOPHILA and Legionella gormanii detected by fluorescent in situ hybridization. Infection, 2004. 32(4): p. 242-5.
20. Stolhaug, A. and K. Bergh, Identification and differentiation of Legionella PNEUMOPHILA and Legionella spp. with real-time PCR targeting the 16S rRNA gene and species identification by mip sequencing. Applied and environmental microbiology, 2006. 72(9): p. 6394-8.
21. Reischl, U., et al., Direct detection and differentiation of Legionella spp. and Legionella PNEUMOPHILA in clinical specimens by dual-color real-time PCR and melting curve analysis. Journal of clinical microbiology, 2002. 40(10): p. 3814-7.
22. Herpers Bjorn, L., et al., Real-time PCR assay targets the 23S-5S spacer for direct detection and differentiation of Legionella spp. and Legionella PNEUMOPHILA. Journal of clinical microbiology, 2003. 41(10): p. 4815-6.
23. Templeton Kate, E., et al., Development and clinical evaluation of an internally controlled, single-tube multiplex real-time PCR assay for detection of Legionella PNEUMOPHILA and other Legionella species. Journal of clinical microbiology, 2003. 41(9): p. 4016-21.
24. Harth, E., et al., Intragenomic heterogeneity and intergenomic recombination among Vibrio parahaemolyticus 16S rRNA genes. Microbiology, 2007. 153(Pt 8): p. 2640-7.
25. Teyssier, C., et al., Atypical 16S rRNA gene copies in Ochrobactrum intermedium strains reveal a large genomic rearrangement by recombination between rrn copies. J Bacteriol, 2003. 185(9): p. 2901-9.
26. Diederen, B. M. and M. F. Peeters, Are oropharyngeal swabs suitable as samples for Legionella-specific PCR testing? J Clin Microbiol, 2007. 45(10): p. 3482; author reply 3482-3.
27. Murdoch, D. R., et al., Use of the polymerase chain reaction to detect Legionella DNA in urine and serum samples from patients with pneumonia. Clin Infect Dis, 1996. 23(3): p. 475-80.
28. Helbig, J. H., et al., Diagnostic relevance of the detection of Legionella DNA in urine samples by the polymerase chain reaction. Eur. J. Clin. Microbiol. INfect Dis, 1999. 18(Oct.): p. 716-722.
29. Matsiota-Bernard, P., S. Waser, and G. Vrioni, Detection of Legionella PNEUMOPHILA DNA in urine and serum samples from patients with pneumonia. Clin. Microbiol. Infect., 2000. 6(4): p. 223-5.
30. Diederen, B. M., et al., Evaluation of real-time PCR for the early detection of Legionella PNEUMOPHILA DNA in serum samples. J Med Microbiol, 2007. 56(Pt 1): p. 94-101.
31. Den Boer, J. W. and E. P. F. Yzerman, Diagnosis of Legionella infection in Legionnaires' disease. European journal of clinical microbiology & infectious diseases—official publication of the European Society of Clinical Microbiology, 2004. 23(12): p. 871-8.
32. Harrison, T. G. and A. G. Taylor, Timing of seroconversion in Legionnaires' disease [letter]. Lancet, 1988. 2: p. 795.
33. Helbig, J. H., et al., Evaluation of the Duopath Legionella lateral flow assay for identification of Legionella PNEUMOPHILA and Legionella species culture isolates. Appl Environ Microbiol, 2006. 72(6): p. 4489-91.

1. A process for detecting a Legionella species comprising:
exposing a sample to a forward primer and a reverse primer under conditions conducive to a polymerase chain reaction to yield an amplicon, wherein each of said forward primer and said reverse primer specific is to a 23S-5S ribosomal intergenetic spacer region of Legionella bacteria; and
detecting said amplicon indicative of the Legionella species.

2. The process of claim 1 wherein said forward primer is SEQ ID NO: 1.

3. The process of claim 1 wherein said reverse primer is SEQ ID NO: 2.

4. The process of claim 1 wherein said probe is specific to Legionella pneumophilia.

5. The process of claim 4 wherein said probe has SEQ ID NO: 3.

6. The process of claim 1 wherein said probe has SEQ ID NO: 4.

7. The process of claim 4 said probe is both SEQ ID NO: 3 and SEQ ID NO: 4.

8. The process of claim 1 wherein said detecting is by a process selected from the group comprising real-time PCR, mass spectrometry, and HPLC.

9. The process of claim 1 wherein said probe is generic to 52 known species of Legionella bacteria.

10. The process of claim 1 wherein yielding said amplicon further comprises performing a cycle of an amplification reaction comprising the steps of denaturing, annealing and extending.

11. The process of claim 1 further comprising annealing said amplicon to said probe during an anneal of said polymerase chain reaction.

12. The process of claim 11 wherein said amplification reaction is a real-time polymerase chain reaction.

13. The process of claim 1 further comprising providing a detectable probe complementary to said amplicon under conditions allowing for said probe to interact with said amplicon such that said detecting is by detection of said interaction between said probe and said amplicon.

14. A process for detecting a Legionella species comprising:
exposing a sample to a forward primer of SEQ ID NO: 1 and a reverse primer of SEQ ID NO: 2 under conditions conducive to a polymerase chain reaction to yield an amplicon;
providing at least one detectable probe complementary to a portion of said amplicon under conditions allowing for said probe to interact with said amplicon; and
detecting said amplicon indicative of the Legionella species.

15. The process of claim 14 wherein said detecting is by using a probe selected from the group comprising of SEQ ID NO: 3 and SEQ ID NO: 4;
hybridizing said probe under conditions suitable for a polymerase chain reaction; and
detecting a detection signal from at least one of said probes.

16-17. (canceled)

18. An isolated nucleotide sequence selected from the group comprising: SEQ ID NO: 1, SEQ ID NO: 2, SEQ ID NO: 3, and SEQ ID NO: 4.

19. The isolated nucleotide sequence of claim 18 wherein said sequence is SEQ ID NO: 4 and wherein said sequence contains locked nucleic acids at positions 3, 6, 8, 10, 13, 14, or combinations thereof.
